(19) United States
(12) Patent Application Publication
(10) Pub. No.: US 2003/0158571 A1
(43) Pub. Date: Aug. 21, 2003

(54) Aortic flow divider for cerebral and coronary embolic protection

(76) Inventors: Brady D. Esch, San Jose, CA (US); John A. Macoviak, La Jolla, CA (US)

Correspondence Address: Gunther Hanke, Fulwider Patton Lee & Utecht, P.O. Box 22615, Long Beach, CA, 90801-5615, US

(21) Appl. No.: 10/077,172

(22) Filed: Feb. 15, 2002

(51) Int. Cl. A61F 2/06

(52) U.S. Cl. 606/192

(57) ABSTRACT

The present invention takes the form of a catheter or cannula having a deployable aortic flow divider mounted on an elongated catheter shaft. The elongated catheter shaft is adapted for introduction into a patient's ascending aorta either by a direct aortic puncture or by a peripheral arterial approach. The aortic flow divider has an undeployed state where it is compressed or wrapped around the catheter shaft and a deployed state where it expands within the aortic lumen. The aortic flow divider is configured to provide embolic protection to the patient's brain and to the coronary arteries of the heart during cardiac surgery and other procedures involving cardiopulmonary bypass or circulatory support. One or more flow channels near the upstream end of the aortic flow divider direct a flow of blood from the superior aortic arch into the aortic root, which creates a washing action that directs potential emboli out of the aortic root and away from the coronary arteries.

### FIELD OF THE INVENTION

The present invention relates generally to an arterial perfusion catheter or cannula for infusion of oxygenated blood or other fluids into a patient for cardiopulmonary bypass or circulatory support. More particularly, it relates to an arterial perfusion catheter with a deployable aortic flow divider for protecting a patient's brain and heart from adverse effects due to embolization that may occur during cardiac surgery and other procedures involving cardiopulmonary bypass or circulatory support.

### BACKGROUND OF THE INVENTION

Over the past decades tremendous advances have been made in the area of heart surgery, including such life-saving surgical procedures as coronary artery bypass grafting (CABG) and cardiac valve repair or replacement surgery. Typically, in order to gain access to the heart a median sternotomy is performed, which creates an open surgical field, conducive for the placement of cannulae and direct visualization for performing the required procedure. Heart activity generally ceases for some period of time, and cardiopulmonary support is provided by diverting blood through an extracorporeal circuit to maintain sufficient oxygenated blood flow to the body and brain while the heart is arrested. Cardiopulmonary bypass (CPB) is a technology that has helped to make these advances possible.

Recently, however, there has been a growing awareness within the medical community, and among the patient population as well, concerning the adverse affects associated with heart surgery, the trauma associated with median sternotomies, as well as well the physiological reactions associated with cardiopulmonary bypass. Chief among these concerns is the potential for stroke or neurologic deficit.

Clinical research has indicated that one of the primary causes of stroke or neurologic deficit is cerebral embolization. Emboli vary in size as well as physical properties and their sources vary. However, embolic materials include atherosclerotic plaques or calcific plaques residing within the ascending aorta or cardiac valves and thrombus or clots from within the chambers of the heart. Emboli may also be dislodged during surgical manipulation of the heart or ascending aorta, aortic cross-clamping, aortic cannulation or due to high velocity jetting from the aortic perfusion cannula (sometimes called the “sandblasting effect”). In addition, air can enter the heart chambers or the blood stream during surgery through open incisions or through the aortic perfusion cannula from the CPB system. Lipid emboli may also enter through the CPB system, particularly when blood salvaged using cardiotomy suction is reintroduced into the circulation. (Brooker R F, Brown W R, Moody D M, et al. Cardiotomy suction: a major source of brain lipid emboli during cardiopulmonary bypass. Annals of Thoracic Surgery, June 1998, 65(6) p1651-5.) As blood is pumped to the brain, either through the extracorporeal circuit or by the beating heart in an off-pump minimally invasive procedure, transient or mobile emboli can become lodged in a vessel of the brain causing a stroke or other neurologic deficit. Clinical studies have shown a correlation between the number and size of emboli passing through the carotid arteries and the frequency and severity of neurologic damage. At least one study has found that frank strokes seem to be associated with macroemboli larger than approximately 100 micrometers in size, whereas more subtle neurologic deficits seem to be associated with multiple microemboli smaller than approximately 100 micrometers in size. In order to improve the outcome of cardiac surgery and avoid adverse neurological effects it would be very beneficial to eliminate or reduce the potential of such cerebral embolic events.

Other devices for embolic protection during cardiac surgery are described in: U.S. Pat. No. 6,254,563 Perfusion shunt apparatus and method, U.S. Pat. No. 6,139,517 Perfusion shunt apparatus and method, U.S. patent application Ser. No. 09/378,676, filed Aug. 20, 1999, Perfusion filter catheter, U.S. patent application Ser. No. 09/158,405, filed Sep. 22, 1998, Aortic catheter with flow divider and methods for preventing cerebral embolization, U.S. patent application Ser. No. 09/447,458, filed Feb. 28, 2001, Cerebral embolic protection assembly and associated methods, and PCT International Patent Application WO 0043062 Aortic catheter with flow divider and methods for preventing cerebral embolization. These patents and patent applications, and all other patents and patent applications referred to herein, are hereby incorporated by reference in their entirety for all purposes. While these previous devices represent a significant advance in technology available for embolic protection during cardiac surgery, there continues to be a need for further research and improvements in this area. In particular, there is a continued need for a device that provides embolic protection to the brain and to the coronary arteries of the heart during cardiac surgery and other procedures involving cardiopulmonary bypass.

The terms downstream and upstream, when used herein in relation to the patient's vasculature, refer to the direction of blood flow and the direction opposite that of blood flow, respectively. In the arterial system, downstream refers to the direction further from the heart along the arterial network, while upstream refers to the direction closer to the heart. The terms proximal and distal, when used herein in relation to instruments used in the procedure, refer to directions closer to and farther away from the operator performing the procedure. Since the present invention is not limited to peripheral or central approaches, the device should not be narrowly construed when using the terms proximal or distal since device features may be slightly altered relative to the anatomical features and the device position relative thereto.

### SUMMARY OF THE INVENTION

In keeping with the foregoing discussion, the present invention takes the form of a catheter or cannula having a deployable aortic flow divider mounted on an elongated catheter shaft. The elongated catheter shaft is adapted for introduction into a patient's ascending aorta either by a direct aortic puncture or by a peripheral arterial approach. The aortic flow divider has an undeployed state where it is pressed against or wrapped around the catheter shaft and a deployed state where it expands within the aortic lumen. The aortic flow divider is configured to provide embolic protection to the patient's brain and the coronary arteries of the heart during cardiac surgery and other procedures involving cardiopulmonary bypass or circulatory support.

Radiopaque markers and/or sonoreflective markers may be located on the catheter and/or aortic flow divider. Preferably, one or more perfusion lumens extend through the elongated catheter shaft to one or more perfusion ports upstream and/or downstream of the aortic flow divider. Oxygenated blood is perfused through the perfusion lumen, or is supplied by the beating heart or a combination of both. Embolic materials that might be dislodged within the heart or ascending aorta are rerouted away from the cerebral circulation by the aortic flow divider.

In use, the aortic flow divider is introduced into the patient's aorta, either by a peripheral arterial approach or by direct aortic puncture, with the aortic flow divider in a collapsed state. The aortic flow divider is advanced across the aortic arch and positioned with the upstream end of the divider in the ascending aorta between the aortic valve and the brachiocephalic artery. The aortic flow divider is then deployed within the aortic arch. When deployed, the aortic flow divider takes on the configuration of a wing or baffle that hemodynamically separates blood flow in the aorta into a first channel that delivers oxygenated blood to the aortic arch vessels and cerebral circulation and a second channel that delivers oxygenated blood to the corporeal circulation. This hemodynamic flow separation reduces the embolic load to the brain by rerouting potential emboli away from the cerebral circulation. In addition, one or more flow channels, preferably located near the upstream end of the aortic flow divider, direct a flow of oxygenated blood from the superior aortic arch into the aortic root, which creates a washing action that directs potential emboli out of the aortic root and away from the coronary ostia.

The position of the catheter and the deployment state of the aortic flow divider may be monitored using fluoroscopy, ultrasound, transesophageal echography (TEE) or aortic transillumination using visible, infrared or near infrared light. Once the aortic flow divider is deployed, oxygenated blood may be infused into the aorta through the perfusion lumen or alternatively the beating heart may supply all the blood or a combination of both. Any potential emboli are rerouted by the aortic flow divider and are thereby prevented from entering the neurovasculature. After use, the aortic flow divider is returned to the collapsed position and the catheter is withdrawn from the patient.

Methods according to the present invention are described using the aortic catheter for partitioning the patient's aortic lumen and performing selective aortic perfusion.

### BRIEF DESCRIPTION OF THE DRAWINGS

FIG. 1 is a perspective drawing of an aortic perfusion catheter having two perfusion lumens with a deployable aortic flow divider shown in the deployed or inflated state.

FIG. 2 is a top view of a distal portion of the perfusion catheter of FIG. 1 with the aortic flow divider shown in the undeployed or deflated state.

FIG. 3 is a cross section of the perfusion catheter of FIG. 1 taken along the line 3-3.

FIG. 4 is a cross section of the perfusion catheter of FIG. 1 taken along the line 4-4.

FIG. 5 is a cross section of the perfusion catheter of FIG. 1 taken along the line 5-5.

FIG. 6 is an exploded view showing the shaft construction of the perfusion catheter of FIG. 1.

FIG. 7 is a cross section of the perfusion catheter of FIG. 1 taken along the line 7-7.

FIG. 8 is a side perspective view of a distal end portion of the aortic catheter of FIG. 1.

FIG. 9 is a cutaway perspective view of a distal end portion of the aortic catheter of FIG. 1.

FIG 10 is a cutaway view of a distal end portion of the aortic catheter of FIG. 1 showing the shaft construction.

FIG 11 shows a flow diagram of the perfusion catheter of FIG. 1 with the aortic flow divider deployed within a patient's aortic arch.

FIG. 12 is a perspective drawing of a perfusion catheter having a single perfusion lumen with a deployable aortic flow divider shown in the deployed or inflated state.

FIG. 13 is a top view of a distal portion of the perfusion catheter of FIG. 12 with the aortic flow divider shown in the undeployed or deflated state.

FIG. 14 is a side view of the perfusion catheter of FIG. 12 with the aortic flow divider shown in an uninflated state positioned alongside an insertable obturator.

FIG. 15 shows a flow diagram of the perfusion catheter of FIG. 12 with the aortic flow divider deployed within a patient's aortic arch.

FIG. 16 shows a flow diagram of a perfusion catheter with an aortic flow divider deployed within a patient's aortic arch via a peripheral artery insertion site.

### DETAILED DESCRIPTION OF THE INVENTION

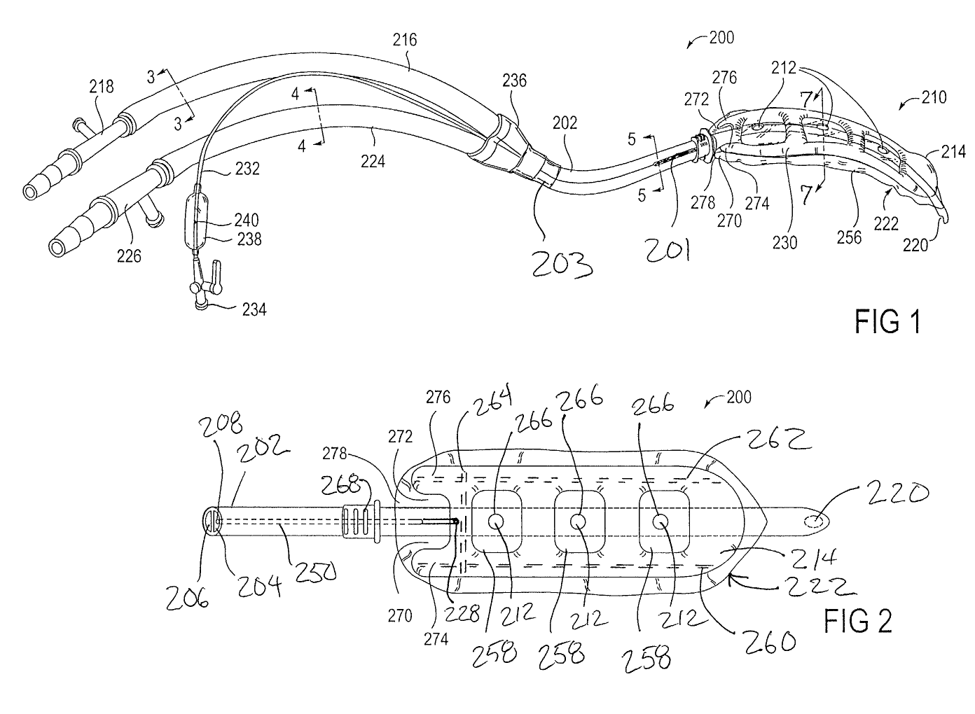

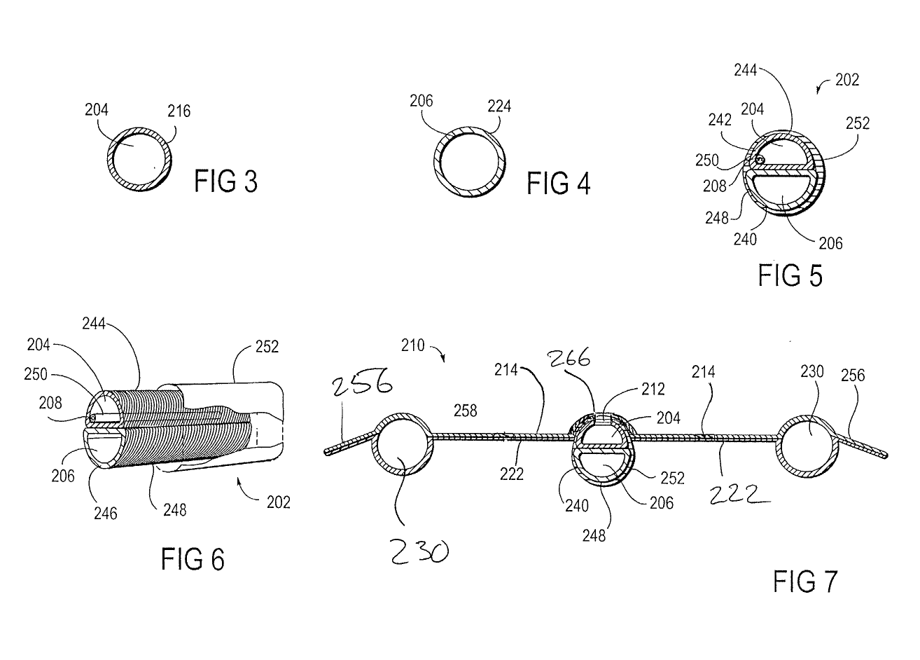

FIGS. 1-11 show an aortic catheter 200 with an aortic flow divider 210 configured for performing differential perfusion of a patient's circulatory system. FIG. 1 shows a perspective view of the aortic catheter 200. In this illustrative example, the aortic catheter 200 is configured for central introduction into the aortic arch through an aortotomy in the ascending aorta. The aortic catheter 200 could alternatively be configured for introduction via peripheral arterial access. The aortic flow divider 210 is mounted on a distal portion of an elongated catheter shaft 202. The catheter shaft 202, shown in cross section in FIG. 5, is constructed with three lumens: an arch perfusion lumen 204, a corporeal perfusion lumen 206 and an inflation lumen 208. The arch perfusion lumen 204 extends through the catheter shaft 202 and communicates on its distal end with one or more arch perfusion ports 212, which are located on an upper surface 214 of the aortic flow divider 210. The proximal end of the arch perfusion lumen 204 connects to an arch perfusion extension tube 216, shown in cross section in FIG. 3, which terminates in an arch perfusion connector 218, such as a barb fitting with a Luer-lock side branch or the like. The corporeal perfusion lumen 206 extends through the catheter shaft 202 and communicates on its distal end with a corporeal perfusion end port 220 and/or corporeal perfusion side ports, which are located near the distal end of catheter shaft 202 and preferably downstream of and below the aortic flow divider 210. Alternatively or in addition, one or more corporeal perfusion ports 220′ may be located near the upstream end of the aortic flow divider 210. The proximal end of the corporeal perfusion lumen 206 connects to a corporeal perfusion extension tube 224, shown in cross section in FIG. 4, which terminates in a corporeal perfusion connector 226, such as a barb fitting with a Luer-lock side branch or the like.

The inflation lumen 208 extends through the catheter shaft 202, preferably within the arch perfusion lumen 204, and connects on its distal end with an inflation port 228, shown in FIGS. 9 and 10, which communicates with the interior of the inflation chamber 230 of the aortic flow divider 210. The proximal end of the inflation lumen 208 connects to, or is continuous with, an inflation lumen extension tube 232, which terminates in an inflation lumen connector 234, such as a stopcock with a Luer-lock connector or the like. A manifold 236, which is preferably an injection molded part, provides the junction where the catheter shaft 202, the arch perfusion extension tube 216, the corporeal perfusion extension tube 224 and the inflation lumen extension tube 232 join together. Optionally, a strain relief tube 203 may be provided to reinforce the junction between the manifold 236 and the catheter shaft 202. Preferably, the aortic catheter 200 includes an inflation indicator 238 on the inflation lumen extension tube 232. The inflation indicator 238 is a small, low-pressure balloon that is mounted on the inflation lumen extension tube 232, such as by heat sealing or adhesive bonding. The interior of the inflation indicator 238 is connected to the inflation lumen 208 by an inflation indicator port 240 on the inflation lumen extension tube 232. Alternatively, the balloon-shaped inflation indicator 238 may be formed integrally with the inflation extension tube 232. The inflation indicator 238 inflates to provide a visual indication whenever the aortic flow divider 210 is inflated.

The catheter shaft 202 may be formed as a multilumen extrusion or it may be formed as a composite construction made up of individual tubes. In one particularly preferred construction, the catheter shaft 202 is constructed by joining together three individual tubes representing the arch perfusion lumen 204, the corporeal perfusion lumen 206 and the inflation lumen 208. FIG. 6 shows an exploded view of the composite construction catheter shaft 202. FIG. 10 shows the catheter shaft 202 with the aortic flow divider 210 removed to illustrate the composite construction more clearly. The corporeal perfusion lumen 206 is constructed as a D-shaped tube 246, which is preferably reinforced over its entire length with a wire coil 248. Similarly, the arch perfusion lumen 204 is constructed as a D-shaped tube 242, which is reinforced over at least part of its length with a wire coil 244. The wire coil 244 reinforcing the D-shaped tube 242 for the arch perfusion lumen 204 preferably extends from the proximal end of the catheter shaft 202 to an intermediate point located under the proximal end of the aortic flow divider 210, and the D-shaped tube 242 continues unreinforced to the distal end of the catheter. A molded plastic tip plug 254 may be inserted into the distal end of the D-shaped tube 242 to terminate and seal the arch perfusion lumen 204 and a second internal plug 255 may be located within the arch perfusion lumen 204 just distal to the most distal arch perfusion port 212. The inflation lumen 208 is constructed as a single lumen tube 250, which, as noted above, may be continuous with the inflation lumen extension tube 232. The three tubes 242, 246, 250 are then covered with a clear, thin-walled tube 252 and heated under pressure to create the composite construction shown in FIG. 5. One or more arch perfusion ports 212 are cut or drilled through the unreinforced wall of the arch perfusion lumen 204 in the distal portion catheter shaft 202.

A gentle S-shaped curve is set into the catheter shaft 202 by placing the catheter shaft 202 on a curved mandrel and heating it. The distal portion of the catheter shaft 202 where the aortic flow divider 210 will be mounted is given a curve that approximates the internal curvature of a human aortic arch. A depth stop 268 is attached to the exterior of the catheter shaft 202 slightly proximal to where the aortic flow divider 210 will be mounted. Preferably, the depth stop 268 is mounted slightly obliquely on the catheter shaft 202, as shown in FIG. 10, so that it will lie flat against the outer wall of the aorta when the curved catheter shaft 202 is inserted through an aortotomy incision into the ascending aorta. Preferably, an orientation stripe 201 or other mark is printed on the exterior of the catheter shaft 202 to indicate the orientation of the aortic flow divider 210 once it has been inserted through the aortotomy incision into the ascending aorta.

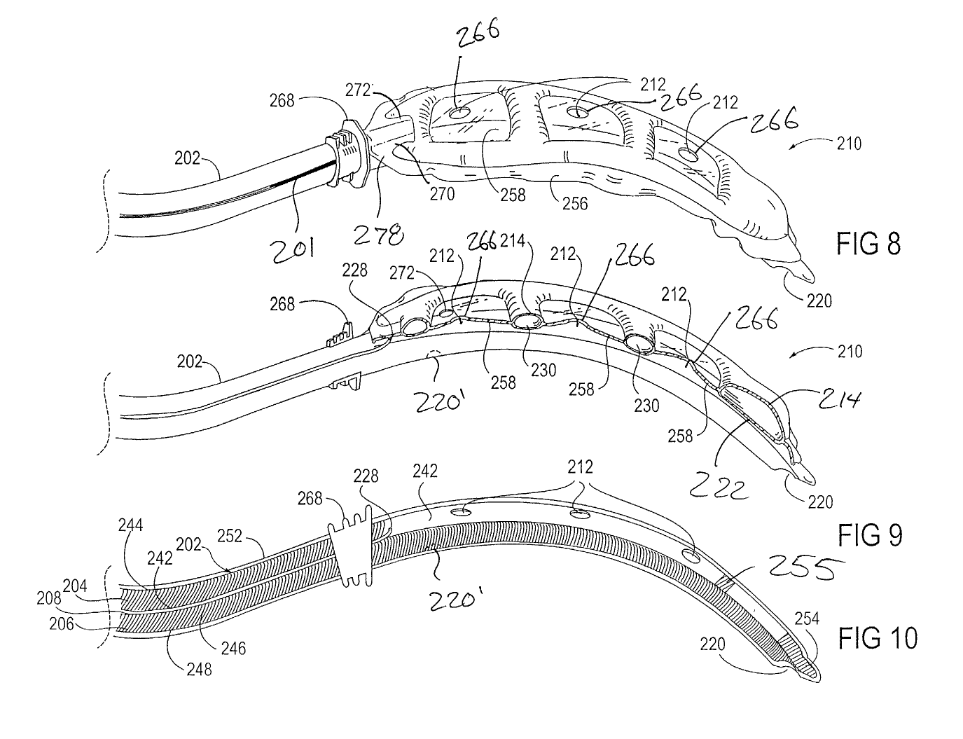

FIG. 2 shows a top view of a distal end portion of the aortic catheter 200 of FIG. 1 showing the aortic flow divider 210 in a deflated condition. FIG. 8 shows a side perspective view of the aortic flow divider 210 in an inflated condition. FIG. 9 shows a cutaway side perspective view of the aortic flow divider 210 in the inflated condition. The aortic flow divider 210 has an upper wall 214 and a lower wall 222 that enclose an inflation chamber 230. The upper wall 214 and lower wall 222 of the aortic flow divider 210 are preferably constructed of a first and second sheet of plastic film that are joined to one another around their peripheral edges 256 and at one or more interior locations 258, for example by heat sealing or adhesive bonding. Suitable materials for the upper wall 214 and lower wall 222 of the aortic flow divider 210 include, but are not limited to, polymers, elastomers, thermoplastics, polyvinylchloride, polyurethane, polyethylene, polyamides, polyesters, silicone, latex, and alloys or copolymers, and reinforced composites thereof. The plastic film that makes up the upper wall 214 and lower wall 222 may have the same or different thicknesses. For example, the upper wall 214 may be made of a thinner plastic film than the lower wall 222.

The aortic flow divider 210 is generally an elongated oval shape that is sized to fit within the lumen of a patient's aortic arch. In one particularly preferred embodiment, the upper wall 214 of the aortic flow divider 210 is slightly larger in length and width than the lower wall 222. When the peripheral edges 256 of the upper wall 214 and lower wall 222 are heat sealed together, this creates a pair of longitudinal folds or wrinkles 260, 262 and at least one lateral fold or wrinkle 264 in the upper wall 214 when the aortic flow divider 210 is deflated, as seen in the top view in FIG. 2. These folds or wrinkles 260, 262, 264 create flow channels that assist the aortic flow divider 210 to deflate fully under applied vacuum.

The interior seals 258 of the aortic flow divider 210 are located so that they will cover the arch perfusion ports 212 in the distal portion of the catheter shaft 202. Holes 266 are cut through the interior seals 258 to coincide with each of the arch perfusion ports 212. Once the aortic flow divider 210 is formed, it is adhesively bonded and/or heat bonded to the distal portion of the catheter shaft 202 with the holes 266 positioned over the arch perfusion ports 212. Alternatively, the holes 266 through the interior seals 258 and the arch perfusion ports 212 may be drilled simultaneously after the aortic flow divider 210 has been bonded to the catheter shaft 202 to assure precise alignment. The distal end of the single lumen tube 250 is connected to the aortic flow divider 210 so that the inflation lumen 208 communicates with the inflation chamber 230 through the inflation port 228.

One or more flow channels 270, 272 are preferably formed near the upstream end of the aortic flow divider 210 to provide a fluid flow path from the upper side to the lower side of the aortic flow divider 210 when it is deployed within a patient's aorta. In the exemplary embodiment shown in FIG. 2, two flow channels 270, 272 are formed between a pair of inflatable tails or extensions 274, 276 extending from the upstream end of the wing-shaped inflatable aortic flow divider 210. The inflatable extensions 274, 276 hold a web 278, which extends between the catheter shaft 202 and the inflatable extensions 274, 276, slightly away from the aortic wall to allow blood to flow through the flow channels 270, 272 into the aortic root.

Prior to use, the aortic flow divider 210 is deflated and pressed against or wrapped around the catheter shaft 202. This reduces the profile of the aortic catheter 200, which facilitates insertion of the aortic catheter 200 through an aortotomy incision or introducer sheath. When it is inflated, the aortic flow divider 210 unwraps or extends from the catheter shaft 202 and assumes a somewhat flattened or gently curved shape that follows the distal curve of the catheter shaft 202. The sealed peripheral edge 256 of the aortic flow divider 210 creates a flexible skirt around the periphery of the aortic flow divider 210 that helps to form a fluid flow seal between the aortic flow divider 210 and the aortic wall.

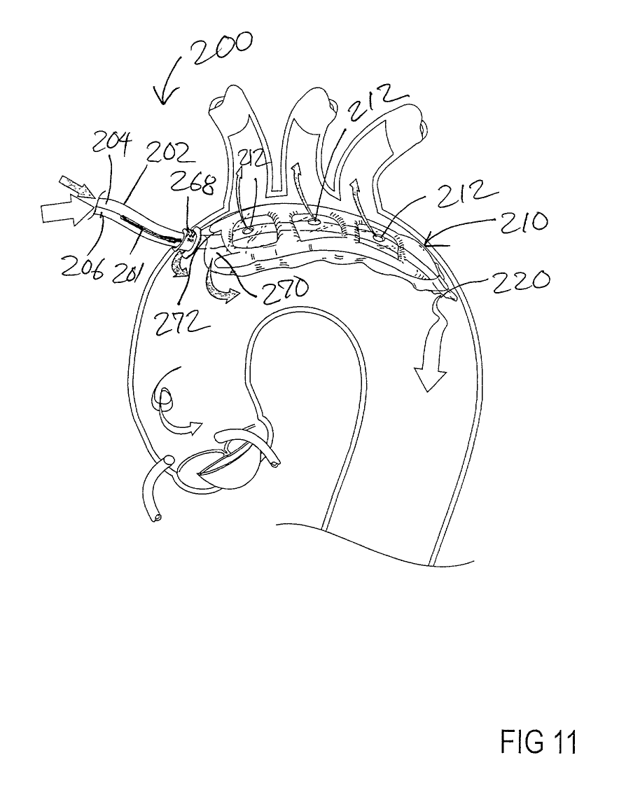

FIG. 11 shows a flow diagram of the perfusion catheter 200 of FIG. 1 with the aortic flow divider 210 deployed within a patient's aortic arch. The patient's corporeal circulation may be perfused with blood or other fluids through the corporeal perfusion lumen 206 and the aortic arch vessels may be separately perfused through the arch perfusion lumen 204. The multiple hole pattern of the arch perfusion ports 212 tends to diffuse the fluid flow exiting the arch perfusion ports 212, which helps to eliminate high velocity jetting that could dislodge plaques, thrombus or other potential embolic materials. In one particularly preferred method, the patient's cerebral circulation is perfused with hypothermic oxygenated blood at approximately 28-34 C. through the arch perfusion lumen 204, while the corporeal circulation is perfused with normothermic oxygenated blood at approximately 35-37 C. through the corporeal perfusion lumen 206. Preferably, the ratio of the flow rates through the arch perfusion lumen 204 and the corporeal perfusion lumen 206 is maintained in the range of approximately 1:2 to 1:4, with a total flow rate of approximately 3-6 liters per minute. Studies have shown that under normothermic conditions, the flow to the arch vessels is approximately 25% of the cardiac output. This percentage drops somewhat as the brain cools to a protective hypothermic state. Maintaining the ratio within this preferred range helps to assure adequate perfusion of the cerebral circulation by providing a flow of oxygenated blood in excess of the demand by the arch vessels. This method creates a hemodynamic flow separation between the cerebral circulation and the corporeal circulation, which protects the brain by redirecting any potential emboli originating in the heart or the ascending aorta toward the corporeal circulation. In addition, excess perfusate from the arch perfusion lumen 204 flows upstream through the flow channels 270, 272 at the upstream end of the aortic flow divider 210 into the aortic root, which creates a washing action that directs potential emboli out of the aortic root and away from the coronary ostia. Optionally, one or more corporeal perfusion ports 220′ positioned on the catheter shaft 202 near the upstream end of the aortic flow divider 210 may provide additional flow into the aortic root to augment this washing action.

The aortic flow divider 210 need not form a perfect seal with the walls of the aorta, nor does it need to be impermeable to emboli, in order to provide cerebral and coronary embolic protection because its primary function is not as a physical barrier to potential emboli. The hemodynamic flow separation between the cerebral circulation and the corporeal circulation provides the primary mechanism for cerebral embolic protection, while the washing action of the aortic root by the flow passing through the flow channels 270, 272 at the upstream end of the aortic flow divider 210 provides the primary mechanism for coronary embolic protection.

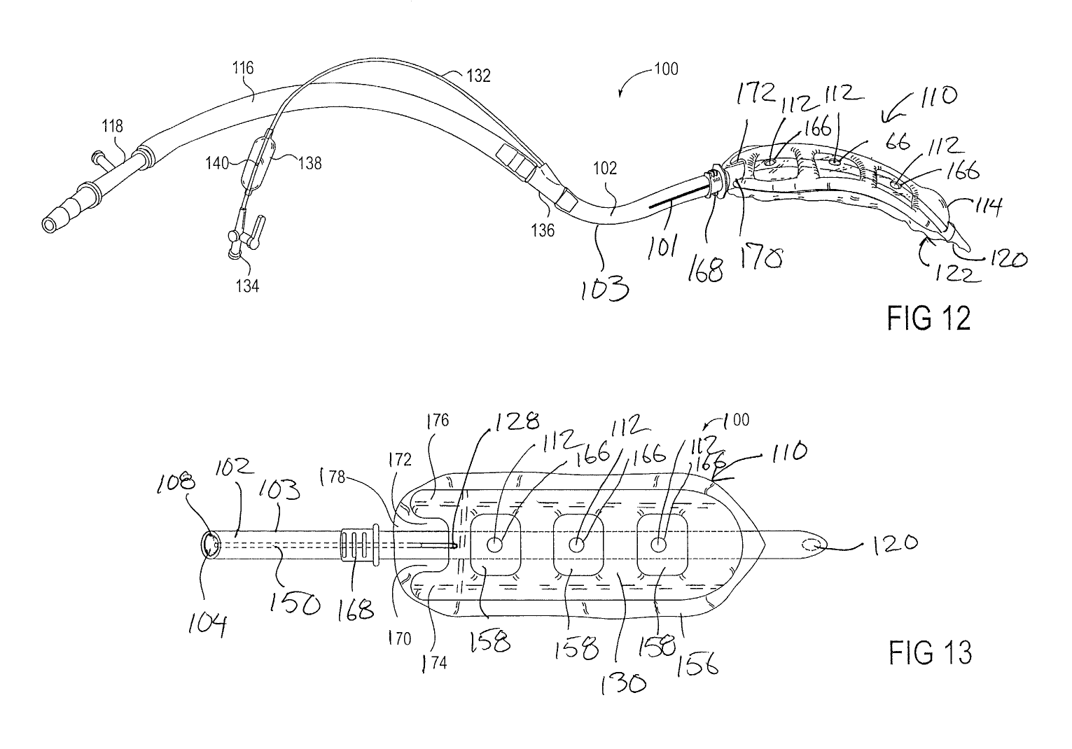

FIGS. 12-15 show an aortic catheter 100 with a aortic flow divider 110 configured for perfusion of a patient's circulatory system. FIG. 12 shows a perspective view of the aortic catheter 100. As in the previous example, the aortic catheter 100 is configured for central introduction into the aortic arch through an aortotomy in the ascending aorta. The aortic catheter 100 could alternatively be configured for introduction via peripheral arterial access. The aortic flow divider 110 is mounted on a distal portion of an elongated catheter shaft 102. The catheter shaft 102 is constructed with two lumens: a perfusion lumen 104 and an inflation lumen 108. The perfusion lumen 104 extends through the catheter shaft 102 and communicates with one or more arch perfusion ports 112, which are located on an upper surface 114 of the aortic flow divider 110, and with a corporeal perfusion end port 120 and/or corporeal perfusion side ports, which are located near the distal end of catheter shaft 102 and preferably downstream of and below the aortic flow divider 110. Alternatively or in addition, one or more corporeal perfusion ports 120′ may be located near the upstream end of the aortic flow divider 110. The proximal end of the perfusion lumen 104 connects to a perfusion extension tube 116 which terminates in a perfusion connector 118, such as a barb fitting with a Luer-lock side branch or the like.

The inflation lumen 108 extends through the catheter shaft 102 and connects on its distal end with an inflation port 128, shown in FIG. 13, which communicates with the interior of the inflation chamber 130 of the aortic flow divider 110. The proximal end of the inflation lumen 108 connects to, or is continuous with, an inflation lumen extension tube 132, which terminates in an inflation lumen connector 134, such as a stopcock with a Luer-lock connector or the like. A manifold 136, which is preferably an injection molded part, provides the junction where the catheter shaft 102, the perfusion extension tube 116 and the inflation lumen extension tube 132 join together. Preferably, the aortic catheter 100 includes an inflation indicator 138 on the inflation lumen extension tube 132. The inflation indicator 138 is a small, low-pressure balloon that is mounted on the inflation lumen extension tube 132, such as by heat sealing or adhesive bonding. The interior of the inflation indicator 138 is connected to the inflation lumen 108 by an inflation indicator port 140 on the inflation lumen extension tube 132. Alternatively, the balloon-shaped inflation indicator 138 may be formed integrally with the inflation extension tube 132. The inflation indicator 138 inflates to provide a visual indication whenever the aortic flow divider 110 is inflated.

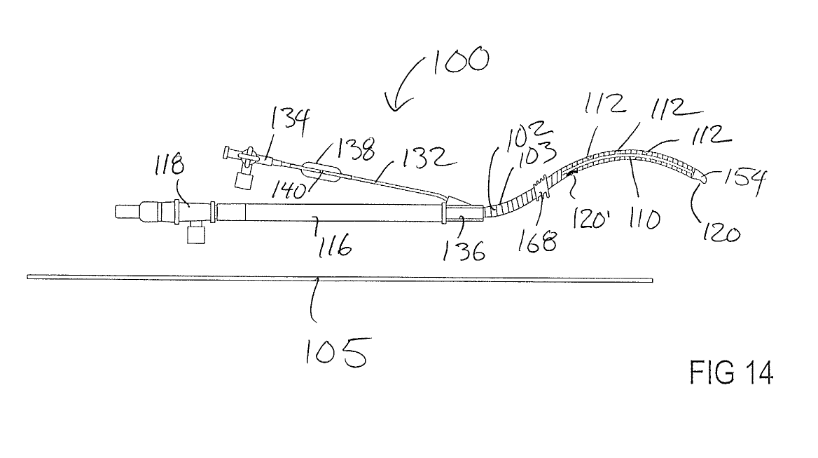

The catheter shaft 102 may be formed as a multilumen extrusion or it may be formed as a composite construction made up of individual tubes. In one particularly preferred construction, the catheter shaft 102 is constructed by joining together two individual tubes representing the perfusion lumen 104 and the inflation lumen 108. The perfusion lumen 104 is preferably constructed as a round cross section tube, which is reinforced over at least part of its length with a wire coil 144, as shown in FIG. 14. A molded plastic tip plug 154 may be inserted into the distal end to terminate the perfusion lumen 104. The inflation lumen 108 is constructed as a single lumen tube, which, as noted above, may be continuous with the inflation lumen extension tube 132. In one particularly preferred embodiment, the tube that forms the inflation lumen 108 passes through the interior of the perfusion lumen 104 in a proximal portion of the catheter shaft 102 that extends from the manifold 136 to the flow divider 110. Optionally, this proximal portion of the catheter shaft 102 may be externally reinforced with a clear, thin-walled heat shrink tube 103 and/or a strain relief tube may be provided to reinforce the junction between the manifold 136 and the catheter shaft 102. One or more arch perfusion ports 112 are cut or drilled through the wall of the perfusion lumen 104 in the distal portion catheter shaft 102.

A gentle S-shaped curve is set into the catheter shaft 102 by placing the catheter shaft 102 on a curved mandrel and heating it. The distal portion of the catheter shaft 102 where the aortic flow divider 110 will be mounted is given a curve that approximates the internal curvature of a human aortic arch. A depth stop 168 is attached to the exterior of the catheter shaft 102 slightly proximal to where the aortic flow divider 110 will be mounted. Preferably, the depth stop 168 is mounted slightly obliquely on the catheter shaft 102, as shown in FIG. 14, so that it will lie flat against the outer wall of the aorta when the curved catheter shaft 102 is inserted through an aortotomy incision into the ascending aorta. Preferably, an orientation stripe 101 or other mark is printed on the exterior of the catheter shaft 102 to indicate the orientation of the aortic flow divider 110 once it has been inserted through the aortotomy incision into the ascending aorta.

FIG. 13 shows a top view of a distal end portion of the aortic catheter 100 of FIG. 12 showing the aortic flow divider 110 in a deflated condition. The aortic flow divider 110 has an upper wall 114 and a lower wall 122 that enclose an inflation chamber 130. The upper wall 114 and lower wall 122 of the aortic flow divider 110 are preferably constructed of a first and second sheet of plastic film that are joined to one another around their peripheral edges 156 and at one or more interior locations 158, for example by heat sealing or adhesive bonding. Suitable materials for the upper wall 114 and lower wall 122 of the aortic flow divider 110 include, but are not limited to, polymers, elastomers, thermoplastics, polyvinylchloride, polyurethane, polyethylene, polyamides, polyesters, silicone, latex, and alloys or copolymers, and reinforced composites thereof. The plastic film that makes up the upper wall 114 and lower wall 122 may have the same or different thicknesses. For example, the upper wall 114 may be made of a thinner plastic film than the lower wall 122.

The aortic flow divider 110 is generally an elongated oval shape that is sized to fit within the lumen of a patient's aortic arch. In one particularly preferred embodiment, the upper wall 114 of the aortic flow divider 110 is slightly larger in length and width than the lower wall 122. When the peripheral edges 156 of the upper wall 114 and lower wall 122 are heat sealed together, this creates a pair of longitudinal folds or wrinkles 160, 162 and at least one lateral fold or wrinkle 164 in the upper wall 114 when the aortic flow divider 110 is deflated, as seen in the top view in FIG. 13. These folds or wrinkles 160, 162, 164 create flow channels that assist the aortic flow divider 110 to deflate fully under applied vacuum.

The interior seals 158 of the aortic flow divider 110 are located so that they will cover the arch perfusion ports 112 in the distal portion of the catheter shaft 102. Holes 166 are cut through the interior seals 158 to coincide with each of the arch perfusion ports 112. Once the aortic flow divider 110 is formed, it is adhesively bonded and/or heat bonded to the distal portion of the catheter shaft 102 with the holes 166 positioned over the arch perfusion ports 112. Alternatively, the holes 166 through the interior seals 158 and the arch perfusion ports 112 may be drilled simultaneously after the aortic flow divider 110 has been bonded to the catheter shaft 102 to assure precise alignment. The distal end of the inflation lumen 108 communicates with the inflation chamber 130 through the inflation port 128.

One or more flow channels 170, 172 are preferably formed near the upstream end of the aortic flow divider 110 to provide a fluid flow path from the upper side to the lower side of the aortic flow divider 110 when it is deployed within a patient's aorta. In the exemplary embodiment shown in FIG. 13, two flow channels 170, 172 are formed between a pair of inflatable tails or extensions 174, 176 extending from the upstream end of the wing-shaped inflatable aortic flow divider 110. The inflatable extensions 174, 176 hold a web 178 of plastic film, which extends between the catheter shaft 102 and the inflatable extensions 174, 176, slightly away from the aortic wall to allow blood to flow through the flow channels 170, 172 into the aortic root.

FIG. 14 shows a side view of the aortic catheter 100 with the aortic flow divider 110 in a deflated condition positioned alongside an insertable obturator 105. The obturator 105 is preferably configured as a flexible rod or tube having a length slightly longer than the overall length of the catheter 100 and a diameter sized to substantially fill the perfusion lumen 104 of the catheter 100. Suitable materials for the obturator 105 include, but are not limited to, polymers, elastomers, thermoplastics, polyvinylchloride, polyurethane, polyethylene, polyamides, polyesters, silicone, latex, and alloys or copolymers, and reinforced composites thereof. The obturator 105 may be inserted into the perfusion lumen 104 of the aortic catheter 100 prior to use in order to reduce backbleeding through the perfusion lumen 104 during insertion of the catheter 100 into the arterial system.

Prior to use, the aortic flow divider 110 is deflated and pressed against or wrapped around the catheter shaft 102. This reduces the profile of the aortic catheter 100, which facilitates insertion of the aortic catheter 100 through an aortotomy incision or introducer sheath. When it is inflated, the aortic flow divider 110 unwraps or extends from the catheter shaft 102 and assumes a somewhat flattened or gently curved shape that follows the distal curve of the catheter shaft 102. The sealed peripheral edge 156 of the aortic flow divider 110 creates a flexible skirt around the periphery of the aortic flow divider 110 that helps to form a fluid flow seal between the aortic flow divider 110 and the aortic wall.

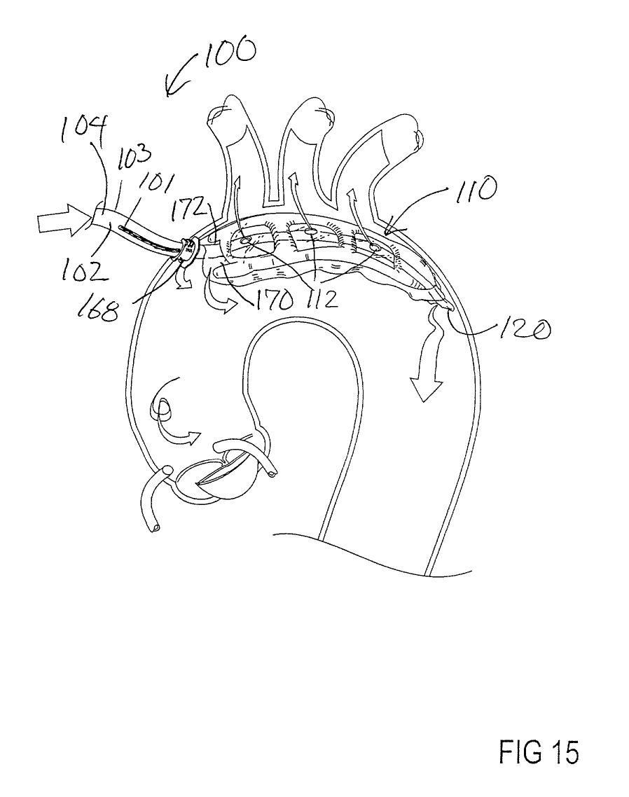

FIG. 15 shows a flow diagram of the perfusion catheter 100 of FIG. 12 with the aortic flow divider 110 deployed within a patient's aortic arch. The patient's circulation may be perfused with oxygenated blood or other fluids through the perfusion lumen 104. Flow from the arch perfusion ports 112 supplies the cerebral circulation, while flow from the corporeal perfusion port 120 supplies the corporeal circulation. The multiple hole pattern of the arch perfusion ports 112 tends to diffuse the fluid flow exiting the arch perfusion ports 112, which helps to eliminate high velocity jetting that could dislodge plaques, thrombus or other potential embolic materials. The patient may be perfused with hypothermic oxygenated blood at approximately 28-34 C. or with normothermic oxygenated blood at approximately 35-37 C. Preferably, the perfusion catheter 100 is configured to provide a flow ratio of approximately 1:2 to 1:4 between the arch perfusion ports 112 and the corporeal perfusion port 120, with a total flow rate of approximately 3-6 liters per minute. Studies have shown that under normothermic conditions, the flow to the arch vessels is approximately 25% of the cardiac output. This percentage drops somewhat as the brain cools to a protective hypothermic state. Maintaining the ratio within this preferred range helps to assure adequate perfusion of the cerebral circulation by providing a flow of oxygenated blood in excess of the demand by the arch vessels. This method creates a hemodynamic flow separation between the cerebral circulation and the corporeal circulation, which protects the brain by redirecting any potential emboli originating in the heart or the ascending aorta toward the corporeal circulation. In addition, excess perfusate from the arch perfusion lumen 104 flows upstream through the flow channels 170, 172 at the upstream end of the aortic flow divider 110 into the aortic root, which creates a washing action that directs potential emboli out of the aortic root and away from the coronary ostia. Optionally, one or more corporeal perfusion ports 120′ positioned on the catheter shaft 102 near the upstream end of the aortic flow divider 110 may provide additional flow into the aortic root to augment this washing action.

As noted above, the aortic flow divider 110 need not form a perfect seal with the walls of the aorta, nor does it need to be impermeable to emboli, in order to provide cerebral and coronary embolic protection because its primary function is not as a physical barrier to potential emboli. The hemodynamic flow separation between the cerebral circulation and the corporeal circulation provides the primary mechanism for cerebral embolic protection, while the washing action of the aortic root by the flow passing through the flow channels 170, 172 at the upstream end of the aortic flow divider 110 provides the primary mechanism for coronary embolic protection.

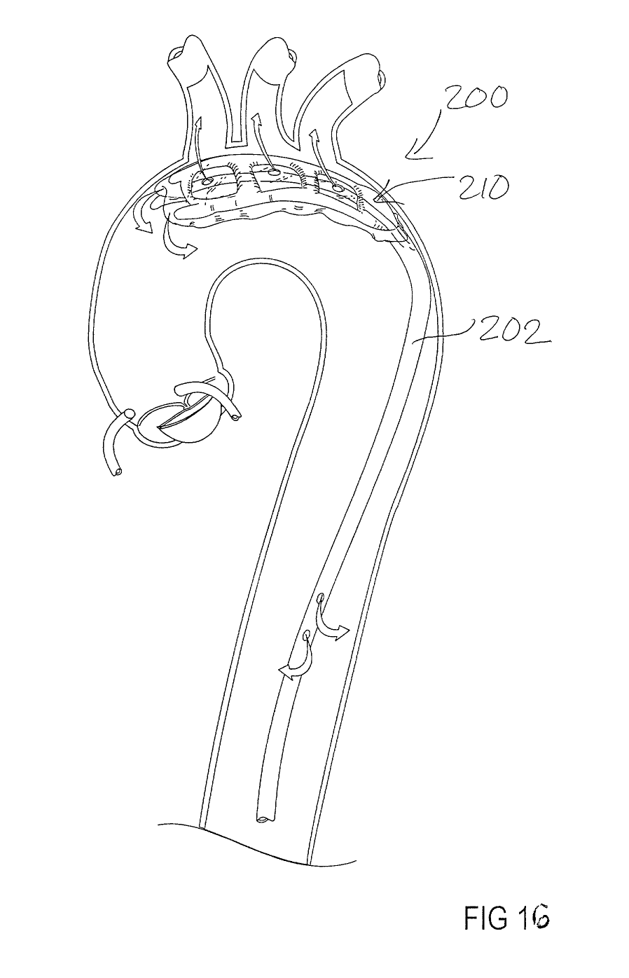

FIG. 16 shows a flow diagram of an exemplary embodiment of a perfusion catheter 200 with an aortic flow divider 210 deployed within a patient's aortic arch via a peripheral artery insertion site. The peripheral entry aortic perfusion catheter 200 of FIG. 16 may be configured with one or two perfusion lumens within the catheter shaft 202.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. An aortic catheter for cerebral and coronary embolic protection, comprising:
an elongated catheter shaft having at least one perfusion lumen extending therethrough;
a deployable aortic flow divider mounted to the catheter shaft; the aortic flow divider having an upper surface and a lower surface, an upstream end and a downstream end; and
at least one flow channel at the upstream end of the aortic flow divider configured to direct fluid flow from the upper surface to the lower surface of the aortic flow divider.

2. The aortic catheter of claim 1, wherein the aortic flow divider comprises an inflatable member.

3. The aortic catheter of claim 2, wherein the catheter shaft comprises an inflation lumen in fluid communication with the inflatable member.

4. The aortic catheter of claim 2, wherein the at least one flow channel is formed between two inflatable extensions extending upstream from the inflatable member.

5. The aortic catheter of claim 2, wherein the at least one flow channel comprises two flow channels adjacent to the catheter shaft at the upstream end of the aortic flow divider.

6. The aortic catheter of claim 3, wherein the two flow channels are formed between two inflatable extensions extending upstream from the inflatable member.

7. The aortic catheter of claim 1, wherein the catheter shaft comprises one perfusion lumen extending from a proximal end of the catheter shaft to at least one arch perfusion port and at least one corporeal perfusion port.

8. The aortic catheter of claim 7, wherein the at least one arch perfusion port discharges above the upper surface of the aortic flow divider and the at least one corporeal perfusion port discharges below the lower surface of the aortic flow divider.

9. The aortic catheter of claim 7, wherein the at least one arch perfusion port and the at least one corporeal perfusion port are configured to provide a fluid flow ratio in the range of approximately 1:2 to approximately 1:4.

10. The aortic catheter of claim 1, wherein the catheter shaft comprises an arch perfusion lumen extending from a proximal end of the catheter shaft to at least one arch perfusion port and a corporeal perfusion lumen extending from the proximal end of the catheter shaft to at least one corporeal perfusion port.

11. The aortic catheter of claim 10, wherein the at least one arch perfusion port discharges above the upper surface of the aortic flow divider and the at least one corporeal perfusion port discharges below the lower surface of the aortic flow divider.

12. The aortic catheter of claim 1, wherein the aortic flow divider is configured to partition the lumen of the aortic arch longitudinally into a first fluid flow channel in fluid communication with the aortic arch vessels and a second fluid flow channel in fluid communication with the patient's corporeal circulation.

13. The aortic catheter of claim 1, wherein the aortic flow divider is configured to divert emboli downstream to the patient's corporeal circulation.

14. The aortic catheter of claim 1, wherein the elongated shaft is sized and configured to be inserted directly into the aorta through an aortotomy incision.

15. The aortic catheter of claim 1, wherein the elongated shaft is sized and configured to be inserted into the aorta through a peripheral artery insertion site.

16. The aortic catheter of claim 1, wherein at least a portion of the elongated shaft is reinforced with a wire coil.

17. A method of cerebral and coronary embolic protection comprising:
inserting an aortic flow divider into an operative position within a lumen of an aortic arch of a patient with an upper surface of the aortic flow divider facing toward the patient's superior aortic arch and a lower surface of the aortic flow divider facing toward the patient's inferior aortic arch;
perfusing the patient's aortic arch vessels through at least one arch perfusion port that discharges into the patient's superior aortic arch; and
directing a portion of the perfusate from the patient's superior aortic arch to the patient's inferior aortic arch through at least one flow channel in the aortic flow divider.

18. The method of claim 17, wherein the aortic flow divider diverts emboli downstream to the patient's corporeal circulation.

19. The method of claim 17, further comprising:
partitioning the lumen of the aortic arch longitudinally into a first aortic flow channel in fluid communication with the aortic arch vessels and a second aortic flow channel in fluid communication with the patient's corporeal circulation.

20. The method of claim 17, wherein the aortic flow divider is inserted directly into the patient's aorta through an aortotomy incision in the aortic wall.

21. The method of claim 17, wherein the aortic flow divider is inserted into the aorta through a peripheral artery insertion site.

22. The method of claim 17, further comprising:
perfusing the patient's corporeal circulation through at least one corporeal perfusion port that discharges into the patient's inferior aortic arch.

23. The method of claim 22, wherein the patient's aortic arch vessels and the patient's corporeal circulation are perfused with a fluid flow ratio in the range of approximately 1:2 to approximately 1:4.

24. The method of claim 22, wherein the patient's aortic arch vessels are perfused with a hypothermic perfusate and the patient's corporeal circulation is perfused with a normothermic perfusate.
